(19) United States
(12) Patent Application Publication
(10) Pub. No.: US 2007/0036729 A1
(43) Pub. Date: Feb. 15, 2007

(54) Compositions containing magnetic iron oxide particles, and use of said compositions in imaging methods

(76) Inventors: Andreas Briel, Berlin (DE); Bernhard Gleich, Hamburg (DE); Juergen Weizenecker, Hamburg (DE); Martin Rohrer, Berlin (DE); Hanns-Joachim Weinmann, Berlin (DE); Hubertus Pietsch, Berlin (DE); Rudiger LaWaczeck, Berlin (DE); Matthias Rothe, Berlin (DE); Jens Thomsen, Berlin (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/476,934

(22) Filed: Jun. 29, 2006

(30) Foreign Application Priority Data
Jun. 29, 2005 (EP) 05014058.1

(51) Int. Cl. A61K 49/10

(52) U.S. Cl. 424/9.34; 977/930

(57) ABSTRACT

The present invention relates to complexes which contain magnetic iron oxide particles in a pharmaceutically acceptable shell, said particles having a diameter of 20 nm to 1 μm with an overall particle diameter/core diameter ratio of less than 6, and to the use of these complexes in magnetic particle imaging (MPI). Particular preference is given to the use of these compositions in examining the gastrointestinal tract, the vascular system of the heart and cranial components, in the diagnosis of arteriosclerosis, infarctions, and tumors and metastases, for example of the lymphatic system.

### INTRODUCTION

The present invention relates to complexes which contain magnetic iron oxide particles in a pharmaceutically acceptable shell, said particles having a diameter of 20 nm to 1 μm with an overall particle diameter/core diameter ratio of less than 6, and to the use of these complexes in magnetic particle imaging (MPI). Particular preference is given to the use of these compositions in examining the gastrointestinal tract, the vascular system of the heart and cranial components, in the diagnosis of arteriosclerosis, infarctions, and tumors and metastases, for example of the lymphatic system.

### PRIOR ART

In imaging methods, particularly in the medical diagnosis sector, contrast agents have led to a considerable improvement in contrast in conventional imaging methods such as X-ray diagnosis and magnetic resonance imaging (MRI). Contrast agents which can be used in MRI can be differentiated on the basis of their mechanism of action (positive amplification or influencing of the longitudinal relaxation or negative amplification or influencing of the transverse relaxation). This effect is expressed as the T1 and T2 relaxivity in the unit mM−1*s−1. R1 and R2 are defined as the steepness of the rise in the curves 1/T1 and 1/T2 against the concentration of the contrast agent. The ratio R2/R1 determines whether a contrast agent has mainly a T1-reducing (R1 significantly higher than R2) or T2-reducing effect (Krombach et al. (2002) Röfo 174: 819-829). Positive contrast agents (also known as relaxivity-increasing or T1-increasing contrast agents) increase the signal strength of perfused areas. Negative contrast agents (also known as susceptibility-increasing or T2-increasing contrast agents) reduce the signal strength of a perfused area in T2-weighted sequences.

Suitable MRI contrast agents have been described in the prior art. By way of example, EP 0 525 199 describes pharmaceutical preparations containing magnetic iron oxide particles which are complexed with polysaccharides, and also the use thereof as contrast agents in MRI. In one preferred embodiment, the superparamagnetic iron oxide cores have a size of 2 nm to 30 nm. The size of the complexes (core plus polysaccharide shell) in suitable preparations is given as 10 nm to 500 nm. However, a specific disclosure extends only to complexes with iron oxide cores having a diameter of 10.1 nm. EP 0 543 020 discloses very similar particles. However, in this case, the superparamagnetic iron oxide particles are complexed with carboxypolysaccharides. The use of these complexes as contrast agents in MRI is also described. The use of carboxydextran as a shell material improves the pharmacological properties of the preparation. In one preferred embodiment, the size of the iron oxide core is 20 nm to 30 nm. However, the specifically disclosed complexes contain only iron oxide cores having a diameter of at most 8.8 nm. U.S. Pat. No. 5,492,814 describes monocrystalline iron oxide particles and also the use thereof to examine biological tissue by means of MRI. Ranges of 1 to 10 nm are specified as the preferred size of the iron oxide cores; however, the examples specifically disclose only particles with iron cores having a diameter of 2.9+/−1.3 nm. In order to improve the suitability as contrast agents in MRI, the disclosed particles are preferably monocrystalline particles, that is to say the crystal structure of the overall particle is homogeneous and free of any disruption—a single crystal.

Recently, a new method for imaging in the medical sector has been described. In this case, the change in the magnetization of particles in a moving magnetic field is measured. This change serves to determine the spatial distribution of the magnetic particles in an examination area (see, for example, DE 101 51 778 A1 and DE 102 38 853 A1). This new technique has been called magnetic particle imaging (MPI). These and other applications by the same applicant mention a number of properties which the particles used in the MPI method must have. By way of example, the particles may be ferromagnetic and ferrimagnetic particles, and thus are similar to the particles known from the MRI method. However, the T1 and T2 relaxivity have no influence on the ability of the particles to be used in MPI. Due to the fundamentally different physical phenomena which are used for imaging in the MRI and MPI methods, the suitability of a particle described in the prior art as a contrast agent for MRI does not determine whether or not the particle is suitable for MPI. Furthermore, it is disclosed that the particles must be so small that only a single magnetic domain (the monodomain) can form therein and no Weiss regions can be produced. It is supposed that, depending on the material, suitable monodomain particles should have an ideal size in the range from 20 nm to approx. 800 nm. Magnetite (Fe3O4) is mentioned as a suitable material for monodomain particles.

MPI is a highly promising new method which is of particular interest in respect of diagnostic applications since the required outlay in terms of apparatus is much lower than in the case of MRI. This is because, unlike in MRI, in MPI there is no need for large homogeneous magnetic fields and therefore the huge superconducting magnets which make MRI diagnosis so expensive and make it difficult for it to be widely used. In order to allow the widespread use of this new technology, however, it is necessary to develop magnetic particles which permit a high spatial resolution, risk-free administration and low magnetic field strengths during the measurement. There is therefore a need to provide particles which are suitable for MPI diagnosis.

### DESCRIPTION OF THE INVENTION

Before the present invention is described in greater detail below, it should be pointed out that this invention is not restricted to the specific methods, protocols and reagents described herein, since these may be varied. The terminology used herein has been used only for the purpose of describing the particular preferred embodiments, and said terminology is not intended to restrict the scope of the invention, the latter being restricted only by the appended claims. Unless otherwise defined, the technical and scientific terms used herein have the meaning which is assigned to them by the person skilled in the art. Preferably, the terms herein are used with the meaning defined in “A multilingual glossary of biotechnological terms: (IUPAC Recommendations)”, Leuenberger, H. G. W, Nagel, B. and Klbl, H. eds. (1995), Helvetica Chimica Acta, CH-4010 Basel, Switzerland).

A number of documents are cited in the description. Each of the documents cited herein (including all patents, patent applications, scientific publications, operating instructions, manufacturers' recommendations, etc.) is fully incorporated herein by way of reference. However, the mention of one of these documents should not in any case be construed as meaning that the present invention can be denied the right based on an earlier date of invention of said publication.

The inventors have now found, surprisingly, that particles which comprise a magnetic iron oxide core which has a particular size and a particular ratio between the diameter of the iron oxide core and the overall particle are particularly suitable for MPI. A first subject matter of the present invention is therefore a particle which comprises, in a pharmaceutically acceptable shell, a magnetic iron oxide core having a diameter of 20 nm to 1 μm with an overall particle diameter/core diameter ratio of less than 6.

It is known that the administration of pure iron oxide cores to patients leads to severe side effects. Blood platelet aggregation and a rapid drop in blood pressure have been described. In order to prevent these side effects, which in some cases may be life-threatening, the iron oxide core of the particle according to the invention is surrounded by a pharmaceutically acceptable shell. A “pharmaceutically acceptable shell” within the context of the present invention is a layer of a substance or a substance mixture which essentially completely encloses the iron oxide core and screens off the iron oxide core in such a way that, when administered to a patient, the known life-threatening side effects do not arise. It is preferred here if the substance or substance mixture is biodegradable, that is to say can be cleaved into small units that can be used by the body and/or can be removed by the kidneys. The particles are administered to the patient preferably in an aqueous colloidal solution or dispersion. It is therefore desirable that the substance or the substance mixture is hydrophilic and prevents the precipitation of the particles and stabilizes the colloidal solution. A plurality of such substances are described in the prior art (see, for example, U.S. Pat. No. 5,492,814).

In one preferred embodiment, the pharmaceutically acceptable shell comprises a synthetic polymer or copolymer, a starch or a derivative thereof, a dextran or a derivative thereof, a cyclodextran or a derivative thereof, a fatty acid, a polysaccharide, a lecithin or a mono-, di- or triglyceride or a derivative thereof. Mixtures of the aforementioned preferred substances are also included.

From these wide substance classes, particular preference is given to the following substances and mixtures thereof:
(i) for polymers or copolymers: polyoxyethylene sorbitan esters, polyoxyethylene and derivatives thereof, polyoxypropylene and derivatives thereof, nonionic surfactants, polyoxyl stearates (35-80), polyvinyl alcohols, polymerized sucrose, polyhydroxyalkyl methacrylamides, lactic acid and glycolic acid copolymers, polyorthoesters, polyalkylcyanoacrylates, polyethylene glycols, polypropylene glycols, polyglycerols, polyhydroxylated polyvinyl matrices, polyhydroxyethyl aspartamides, polyamino acids, styrene and maleic acid copolymers, polycaprolactones, carboxypolysaccharides, and polyanhydrides;
(ii) for starch derivatives: starch 2-hydroxymethyl ether and hydroxyethyl starch;
(iii) for dextrans or derivatives thereof: galactosylated dextrans, lactosylated dextrans, aminated dextrans, dextrans containing SH groups, dextrans containing carboxyl groups, dextrans containing aldehyde groups, biotinylated dextrans;
(iv) for cyclodextrins: beta-cyclodextrins and hydroxypropyl cyclodextrins;
(v) for fatty acids: sodium lauryl sulfates, sodium stearates, stearic acids, sorbitan monolaurates, sorbitan monooleates, sorbitan monopalmitates and sorbitan monostearates.

Due to its particularly good compatibility, preference is given to the use of dextrans and polyethylene glycol (PEG) and derivatives thereof and in particular carboxydextrans, low-molecular-weight PEGs (preferably 500 to 2000 g/mol) or high-molecular-weight PEGs (more than 2000 g/mol to 20,000 g/mol) in order to form the pharmaceutically acceptable shell. In one particularly preferred embodiment, the pharmaceutically acceptable shell is biodegradable. A plurality of the preferred substances and polymers disclosed above satisfy this property.

In the prior art, a plurality of methods are known which are suitable for producing iron cores with a shell. In some of the methods, the iron core is formed in a first method step and the shell is applied in a further step. However, it is also possible to form the iron core and the shell in one reaction or in a “one-pot reaction”. Known methods include, without limitation, the aerosol/steam pyrolysis method, the chemical vapor deposition method, the sol-gel method and the microemulsion method. Particularly preferred methods for creating a shell for iron oxide cores have been described for example in EP 0 543 020 B 1, EP0 186 616, EP 0 525 199 and EP0 656 368.

It is preferred if the magnetic iron oxide core comprises magnetite or maghemite or mixtures thereof. It is possible that, in some embodiments, further metal oxides are added to the iron oxide core, said metal oxides preferably being selected from magnesium, zinc and cobalt. These further metal oxides may be added to the iron oxide core in proportions of up to 20% in total. Furthermore, it is also possible for manganese, nickel, copper, barium, strontium, chromium, lanthanum, gadolinium, europium, dysprosium, holmium, ytterbium and samarium to be contained in quantities of less than 5%, preferably less than 1%.

The overall diameter of the particles according to the invention depends on the diameter of the iron oxide core and on the thickness of the pharmaceutically acceptable shell surrounding the latter, and also on any molecules attached to the surface of the shell. The upper limit of the overall diameter is defined by the proviso that the particles must be able to pass through the capillaries following application to the body of a patient. The capillaries with the smallest diameter are usually located in the lungs. These capillaries can still be passed through by particles having an overall diameter of 2 μm. Particles according to the invention are preferably spherical. However, particles which are elongate or angular or essentially of any shape are also covered by the invention, provided that the surface of the iron core is essentially surrounded by the pharmaceutically acceptable shell. Thus, the overall diameter of a particle is 2r in the case of a spherical particle, and in the case of a particle of irregular shape is defined by the distance between the two points on the particle surface which lie furthest away from one another, plus the thickness of the hydrated shell. The overall diameter defined here must be distinguished from the mean overall diameter of a particle according to the invention, which is defined as the average distance of all points on the surface of the particle from the center of gravity of the particle, plus the thickness of the hydrated shell. The mean overall diameter is in turn distinguished from the averaged overall diameter of the particles, which refers to a group of particles and is obtained as the average value of all the mean particle diameters of the particles contained in the group, plus the hydrated shell. The lower limit of the overall diameter is determined by the lower limit of the diameter of the iron oxide core, which leads to improved imaging in the MPI method. In one preferred embodiment, the overall particle diameter therefore varies within a range from approx. 30 nm to approx. 2 μm. However, even more preference is given to the use of particles having an overall diameter in a range from approx. 40 nm to approx. 500 nm, more preferably in a range from approx. 45 nm to approx. 300 nm, even more preferably in a range from approx. 50 nm to approx. 200 nm. The overall diameter of the particles according to the invention can be determined by a number of direct and indirect methods known in the prior art, which include for example electron microscopy and dynamic light scattering. Preferably, the overall diameter is determined by dynamic light scattering.

In the available prior art, and above all in the aforementioned documents DE 101 51 778 A1 and DE 102 38 853 A1, particles are described which the applicant assumes to be suitable for MPI. Said prior art discloses only that particles which have a magnetic core having a diameter of 20 to 800 nm are suitable, and that the actually suitable diameter of the magnetic core depends on the respectively selected magnetic material. There is no teaching which discloses the use of particles which have iron oxide cores having a diameter of 20 nm to 1 μm with an overall particle diameter/core diameter ratio of less than 6. Thus, preferred lower limits of the diameter of the iron oxide core are 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 85, 90, 95, 100 nm. Preferred upper limits of the diameter are 500, 490, 480, 470, 460, 450, 440, 430, 420, 410, 400, 390, 380, 370, 360, 350, 340, 330, 320, 310, 300, 290, 280, 270, 260, 250, 240, 230, 220, 210, 200, 190, 180, 170, 160, 150, 140, 130, 120, 110, 100, 95, 90, 85 and 80 nm. All combinations of the abovementioned upper and lower limits are possible in order to define the preferred range for the particle size, provided that the value of the upper limit is greater than the value of the lower limit, for example 40 to 400 nm, 50 to 200 nm, etc. Preferred particles according to the invention have an iron oxide core diameter in a range from approx. 25 nm to approx. 500 nm. Even more preferably, the diameter of the iron oxide core lies in a range from approx. 30 nm to approx. 200 nm, yet more preferably from approx. 35 nm to approx. 80 nm. The diameter of the core can once again be determined by methods known in the prior art, which include, without limitation to the methods mentioned, X-ray structural analysis and electron microscopy. Since the iron oxide cores do not in all cases have a spherical shape, the diameter of the iron oxide core is the distance between the points on the surface of the iron oxide core which lie furthest away from one another. This diameter must be distinguished from the mean diameter of an iron oxide core, which is defined as the average distance of all points on the surface of the iron oxide core from the center of gravity of the iron oxide core. The mean diameter of an iron oxide core is in turn distinguished from the averaged diameter of the iron oxide core, which refers to a group of particles and is obtained as the average value of all the mean diameters of the iron oxide cores contained in the group.

It has surprisingly been found that particles which have a thin shell of a pharmaceutically acceptable substance around the iron oxide core are more suitable for MPI than particles which have a thicker shell while having the same iron oxide core diameter. In one preferred embodiment, the particles according to the invention are therefore characterized in that the ratio of the overall particle diameter to the iron oxide core diameter is less than 6. Even more preferably, this ratio is less than 5.5, 5.0, 4.5, 4.0, 3.5, 3.0, 2.9, 2.8, 2.7, 2.6, 2.5, 2.4, 2.3, 2.2, 2.1, 2.0, 1.95, 1.90, 1.85, 1.80, 1.75, 1.60, 1.55 or less than 1.5. With regard to the aforementioned diameters of the overall particles and iron oxide cores, particularly preferred particles have iron oxide cores having a diameter in the range from approx. 30 to approx. 200 nm and even more preferably in the range from approx. 35 nm to approx. 80 nm with a ratio of overall particle diameter to iron oxide core diameter which is less than 3.0, 2.9, 2.8, 2.7, 2.6, 2.5, 2.4, 2.3, 2.2, 2.1, 2.0, 1.95, 1.90, 1.85, 1.80, 1.75, 1.60, 1.55 or less than 1.5.

In connection with the present invention, the term “polycrystalline magnetic iron oxide particles” refers to magnetic iron oxide particles which consist of at least 2 coherent crystals, preferably of 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100 or more crystals. The maximum number of crystals which can be contained in one iron oxide particle of the invention is limited only by the size of the particle. More crystals can be contained in larger particles than in smaller particles. The crystals which are contained in the polycrystalline magnetic iron oxide particles preferably have a length in a preferred direction of 1-100 nm, preferably 3 to 50 nm. One unit cell of a magnetite crystal (Fe3O4) has an edge length of approx. 1 nm. Accordingly, the crystals which are contained in the polycrystalline magnetic iron oxide particles have, along a preferred direction, preferably at least 3 unit cells, even more preferably 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, 100 or more unit cells. In the polycrystalline iron oxide cores, non-crystalline areas with an unordered amorphous structure may be formed at the interfaces between one or more crystals, that is to say polycrystalline magnetic iron oxide cores preferably consist of 50%, even more preferably 55%, 60%, 65%, 70%, 75%, 80%, 85%, 90%, 95% or more, of polycrystalline areas and the rest of the iron oxide particle consists of unordered amorphous areas. The individual crystals contained in the iron oxide core may have the same or different lengths. The largest crystallite, that is to say individual crystal, which is contained in the polycrystalline magnetic iron oxide particles should preferably have a volume that is less than 70%, even more preferably less than 65%, less than 60%, less than 55%, less than 50%, less than 45% of the total volume of the particle according to the invention. In one embodiment of the invention, there is no long-range order in the polycrystalline iron oxide core, but rather only a short-range order. This means that the particle is in the form of an essentially amorphous particle with crystalline inclusions. In the context of the present invention, “polycrystalline” covers both iron oxide particles with a long-range order and those with a short-range order. Crystals with a long-range order are preferred.

It should be pointed out that polycrystalline iron oxide cores can also form a monodomain and do not necessarily lead to the formation of Weiss regions. The size of the iron oxide core is a critical factor in terms of the ability to form Weiss regions. In one particularly preferred embodiment, the iron oxide core contains at least five iron oxide monocrystals. The number and size of the crystals contained in a particle can be detected by means of a plurality of different methods which include, without limitation, transmission electron microscopy (TEM), electron tomography and X-ray diffraction. Preferably, the size is determined by TEM.

MPI is based on detecting the position of magnetic particles. In diagnostic methods which are aimed only at showing the internal structure of areas that are flowed through by fluid, such as showing the gastrointestinal tract or showing the coronary arteries for example, it may be sufficient to provide the patient with a sufficient quantity of the particles, for example by injecting a particle dispersion or by swallowing a suitable particle solution or dispersion. The particles, which essentially do not bind to structures in the area examined, can then be observed as they flow through the examined area. However, for many diagnostic applications, it is desirable that the particles exhibit a specific affinity for surface structures of the areas examined. Therefore, in one preferred embodiment, the particle according to the invention comprises one or more identical or different ligands on its surface. The ligands may be covalently or non-covalently bonded to the surface. Within the context of the present invention, a “ligand” is a substance which binds to a given substance with an IC50 of less than 10 μM, preferably of less than 1 μM, less than 900 nM, less than 800 nM, less than 700 nM, less than 600 nM, less than 500 nM, less than 400 nM, less than 300 nM, less than 200 nM, less than 100 nM, less than 90 nM, less than 80 nM, less than 70 nM, less than 50 nM, less than 40 nM, less than 30 nM, less than 20 nM. In the prior art, a plurality of methods are known for determining the binding affinity of a ligand (IC50 or some other parameter) to a given substance. These methods include, without limitation, ELISA, surface plasmon resonance and radioligand binding assay methods, as described for example in Gazal S. et al (2002) J. Med. Chem. 45: 1665-1671.

In some cases, it may be desirable to immobilize two or more different ligands on the surface of a particle according to the invention. This is possible for example when, on the surface to which the particle is to specifically bind, there are two structures characteristic of this surface arranged adjacent to one another. This may be the case for example in certain tumor cells in which, due to a mutation, two components of a receptor are permanently joined to one another. The use of two ligands which are each directed against one of the two structures then leads to a considerable increase in affinity or specificity. In this preferred embodiment of the particles according to the invention, additionally one or more types of ligand is (are) immobilized on the surface of the shell, which consists of a pharmaceutically acceptable substance. Depending on the respective substance or substance mixture of which the pharmaceutically acceptable shell consists, the latter may itself have affinity for a given substance. By way of example, a number of polysaccharides have a certain cell specificity. The production of e.g. cell-specific particles therefore does not in all cases require the immobilization of ligands, since such an affinity may also result from the pharmaceutically acceptable shell.

The maximum number of ligands that can be immobilized on the surface is determined by the size of the surface and the space required by the respective ligand. Usually, the ligand is bound to the surface of the particle in a monomolecular layer which, depending on the size of the ligand, may lead to a significant increase in diameter. When selecting suitable ligands, and in particular when using large antibody ligands, care must therefore be taken to ensure that the overall diameter of the resulting ligand-coated particle does not lead to the situation whereby the particle is no longer able to pass through the capillaries. It is therefore preferred that the diameter of the ligand-coated particle is less than 2 μm, and even more preferably has a diameter which is said to be preferred with regard to the overall diameter of the uncoated particle as mentioned above. In this case, the thickness of the pharmaceutically acceptable shell and/or the diameter of the iron oxide core must accordingly be reduced. In the prior art, a plurality of methods for attaching ligands are disclosed. Particularly preferred methods are disclosed for example in U.S. Pat. No. 6,048,515. Depending on the respective pharmaceutically acceptable shell, it may be necessary to crosslink the shell before attaching the ligand. Such methods have been described for example by Weiβleder et al.

The choice of ligand will depend on the disease or condition which is to be diagnosed by MPI. Preferably, the structures to which the ligands located on the particles bind are contained in the areas flowed through by body fluids, for example blood or lymph, or are contained in the body fluids. It is therefore preferred that the ligand is able to bind specifically to cellular (eukaryotic or prokaryotic), extracellular or viral surface structures. In the prior art, a plurality of structures are known which are preferentially expressed in diseased tissues or cells or in the vicinity of such tissues or cells and which can therefore serve as an indication of the respective disease. By way of example, the new formation of blood vessels (neoangiogenesis) in the adult body is restricted to the endometrium in connection with menstruation or pregnancy and to healing processes following vascular trauma. However, it is known that new blood vessels are also formed in a plurality of proliferative diseases, and said new blood vessels are not found at any other point in the body which is not affected by the proliferative disease. Therefore, cellular structures which are produced in connection with neoangiogenesis, and in particular structures which are found only on tumor endothelium, such as for example the ED-B domain of fibronectin (ED-BF), are excellent targets for the ligands which can be immobilized on the surface of the particles according to the invention. All the ligands known in the prior art against such structures associated with diseases can be used in conjunction with the particles of the present invention. However, particularly preferred ligands are the ligands which are able to bind specifically to one of the following structures: to the ED-B domain of fibronectins (ED-BF), to endoglin, to the vascular endothelial growth factor receptor (VEGFR), to members of the VEGF family, to NRP-1, to Ang1, to Thie2, to PDGF-BB and receptors, to TGF-β1, to TGF-β receptors, to FGF, to HGF, to MCP-1, to integrins (αvβ3, αvβ5, α5β1), to VE-cadherins, to PECAM (CD31), to ephrins, to plasminogen activators, to MMPs, to PAI-1, to NOS, to COX-2, to AC133, to chemokines, to Id1/Id3, to VEGFR-1, to Ang2, to TSP-1, -2, to angiostatins and related plasminogen kringles, to endostatins (collagen XVII fragment), to vasostatin, to platelet factor 4 (PF4), to TIMPs, to MMP inhibitors, to PEX, to Meth-1, to Meth-2, to IFN-α, -β, -γ, to IL-10, to IL-4, to IL-12, to IL-18, to prolactin (M, 16K), to VEGI, to fragments of SPARC, to osteopontin fragments or maspin, to CollXVIII, to CM201, to statins, in particular L-statin, to CD105, to ICAM1, to somatostatin (subtype 1, 2, 3, 4 or 5) or somatostatin receptors (subtype 1, 2, 3, 4, 5 or 6).

It is known in the prior art that a plurality of substances have an affinity preferably for cellular (eukaryotic or prokaryotic), extracellular or viral surface structures. Preferably, the ligands which are immobilized on the particles according to the invention are selected from a polypeptide, an oligonucleotide, a polysaccharide and a lipid.

Polypeptides which have a specific affinity are known and can be identified by a number of methods including “phage display” and immunization. In this connection, it is preferred if the proteins which according to the invention can be used as ligands are selected from the group consisting of an antibody, comprising human, humanized and chimeric antibodies and antibody fragments, fragments comprising antibody binding domains, for example Fv, Fab, Fab′, F(ab′)2, Fabc, Facb, single-chain antibodies, for example single-chain Fvs (scFvs) and diabodies, and a ligand of a cellular, extracellular or viral receptor or a fragment thereof. Suitable ligands are for example the Vascular Endothelial Growth Factor (VEGF), Epidermal Growth Factor (EGF), chemokines or cytokines.

The ability of nucleic acids to enter into specific bonds for example with transcription factors or histones is well known. Preferred oligonucleotides which can be immobilized as ligands on the particles according to the invention include DNA, RNA, PNA and aptamers. Particular preference is given to PNAs, since these have a higher resistance to the nucleases usually found in patients and thus have a longer biological half-life. Methods for identifying specifically binding nucleic acids are known in the prior art and are described for example in WO 93/24508 A1, WO 94/08050 A1, WO 95/07364 A1, WO 96/27605 A1 and WO 96/34875 A1.

In some cases, for example for reasons of steric or chemical incompatibility, it will not be possible to bind the ligand directly to the surface of the particle. In these cases, the ligand can be bound to the surface of the particle via a linker. In this connection, the term “linker” denotes a molecule which preferably has one or two chemically reactive groups which respectively permit covalent or non-covalent coupling to the particle surface on the one hand and to the ligand on the other hand. Between these coupling groups, there is usually a linear, cyclic or branched region which allows for example greater spatial separation between the ligand and the particle and a greater mobility of the ligand. This linear region may be for example a substituted or unsubstituted, branched or unbranched, saturated or unsaturated alkyl chain (C2 to C50), which may be interrupted by one or more O, N and/or S atoms, or a polpeptide or a polynucleotide. Examples of chemically reactive groups which can be used in these linkers include, for example, amino, hydroxyl, thiol or thiol-reactive, sulfhydryl, carboxyl and epoxide groups. Thiol-reactive groups include for example maleinimide (maleimide), chloroacetyl, bromoacetyl, iodoacetyl, chloroacetamido, bromoacetamido, iodoacetamido, chloroalkyl, bromoalkyl, iodoalkyl, pyridyl disulfide and vinylsulfonamide groups. A plurality of coupling reagents, coupling groups and linkers are disclosed in WO 98/47541, to which specific reference is made here with regard to this disclosure.

It has been found that polyethylene glycol residues (PEG) and/or polypropylene glycol residues (PPG) which are immobilized on the surface of pharmaceutical active agents lead to a considerable lengthening of the biological half-life. Examples of this are PEGylated liposomes or PEGylated proteins, such as. PEGylated EPO for example. Methods for the PEGylation of surfaces are well known in the prior art. Depending on the respective pharmaceutically acceptable shell material that is used, the PEG residues or the PPG residues may be covalently or non-covalently immobilized on the surface directly or via a linker. Preferably, the polyethylene glycol and/or polypropylene glycol residues are non-covalently bonded to the surface of the particle.

Another subject matter of the present invention is a method for producing particles which comprise, in a pharmaceutically acceptable shell, a magnetic iron oxide core having a diameter of 20 nm to 1 μm and with an overall particle diameter/core diameter ratio of less than 6, said method comprising the following steps:
(i) mixing an aqueous mixed iron salt solution, which contains an iron(II) salt and an iron(III) salt, with a base in the presence of a synthetic polymer or copolymer, a starch or a derivative thereof, a dextran or a derivative thereof, a cyclodextran or a derivative thereof, a fatty acid, a polysaccharide, a lecithin or a mono-, di- or triglyceride or a derivative thereof or mixtures thereof, until a colloidal solution of the particles is formed,
(ii) passing the particle-containing solution through a magnetic gradient field,
(iii) removing the magnetic gradient field,
(iv) and recovering the particles retained in the gradient field.

Step (i) of the method according to the invention usually does not lead to a homogeneous group of particles but rather to a group of particles which vary within certain bandwidths both with regard to the diameter of the iron oxide core and with regard to the diameter of the overall particle. The averaged iron oxide core diameter of such a group is obtained as the average value of all the mean iron oxide core diameters contained in the group. Application of the magnetic gradient field in step (ii) leads to the selection of particles with an iron oxide core that is larger than the averaged iron oxide core diameter. For example, by selecting the strength of the magnetic gradient field and possibly by repeating steps (ii) to (iv) of the method according to the invention one, two or more times, possibly while increasing the strength of the gradient field, it is possible to select, from a heterogeneous group of particles, a group of particles which have a larger averaged iron oxide core diameter than the averaged iron oxide core diameter and a thinner shell of pharmaceutically acceptable materials. Usually, a particle group which has a smaller scattering of the core diameter is also obtained at the end of steps (ii) to (iv).

In order to select, from this subgroup of particles, the particles which have at least a predefined overall particle diameter, these can be selected by methods known in the prior art, such as filtration, sedimentation, counter-current centrifugation (elutriation), etc.

The iron(II) and iron(III) salts used in the method according to the invention are preferably in aqueous solution at a concentration of 0.1 to 2 M. The divalent and trivalent iron ions are preferably in a mixing ratio of 1:3 to 2:1. Suitable anions of the iron salts are derived from organic acids such as, for example, from citric acid, lactic acid, acetic acid, maleic acid, etc., or from inorganic acids such as, for example, from HCl, H2SO4, H2SO3, HBr, HI, HNO3 or HNO2.

The base is preferably selected from inorganic bases such as, for example, NaOH, KOH, LiOH or Al(OH)₃ and from organic bases. The base may be added to the aqueous reaction solution as a solid or as a solution, preferably as an aqueous solution. This addition preferably takes place until the solution reaches a pH of 10, preferably 11 or higher. This step of basification is preferably followed by a neutralization by means of acid, preferably HCl, to a pH of approximately 7±0.5.

As a further step, step (i) may be followed directly, or after neutralization, by a heat treatment. Here, the solution is heated to at least 50° C., preferably to 60° C., 65° C., 70° C., 75° C., 80° C., 90° C. or 95° C. If no neutralization has yet taken place, the solution can be neutralized by adding acid after heating, and then can either be cooled or heated further. If the solution is heated further, it is preferably heated to 50° C., more preferably to 60° C., 65° C., 70° C., 75° C., 80° C., 85° C. 90° C., 95° C., 100° C. or to reflux. Such heating preferably takes place for 10 min to 10 h.

Directly after step (i) or else following each of the further steps described above, that is to say the neutralization and/or heating step, one or more washing and/or dialysis steps may be carried out. Furthermore, it is preferred if the particles obtained after step (iv) are subjected to one or more washing and dialysis steps. The aim of this step (or these steps) is preferably to separate from the particles any potentially damaging impurities left over from the production process, and to adapt the pH and/or the salt content of a solution containing the particles.

The magnetic gradient field is used to select particles which are particularly suitable for the MPI method. The magnetic gradient field which can be used for the method according to the invention can vary widely and can be set by the person skilled in art taking into account various parameters of the test arrangement. The gradient field used for separation purposes must be considerably greater than the gradient of the terrestrial field. The magnetic gradient field in the separation chamber may be generated by a permanent magnetic material or by a conductor which is flowed through by current. In connection with the present invention, the latter embodiment is preferred since it makes it easy to remove the gradient field by switching off the current. In the first case, the magnetic gradient field is removed by removing the permanent magnetic material. The gradient field is typically in the range from 1 mT/m to 5000 T/m. If, for example, particles having a core diameter>100 nm are to be retained, and if step (ii) is carried out on an arrangement having a low throughput rate, it is preferable to use gradient strengths of 1-10 mT/m. However, if particles having a smaller core diameter of for example approx. 20 nm are to be retained, and if the method is carried out with a high throughput rate, it is preferable to use gradient strengths>1000 T/m. The aim of applying a gradient field consists in concentrating those particles which have a relatively large iron oxide core diameter and/or which have a preferred, that is to say low, overall diameter/core ratio. Taking account of the teaching provided here, and depending on the averaged diameter, the particles produced by the method according to the invention and the flow rate used, the person skilled in the art can determine a suitable gradient field strength which makes it possible to select such particles. Suitable separation devices provided with a gradient field are described by way of example below in Examples 1b) and 1c) and in EP 0 915 738 B1. The devices may contain further paramagnetic materials in the region of the gradient field, in order if necessary to increase the gradient field.

In order to recover the particles retained in the gradient field, after the gradient field has been removed, the separation device is preferably rinsed through with a suitable solution, preferably an aqueous pharmaceutically acceptable solution, wherein the particles are preferably released mechanically from the separation device.

The particles produced according to the invention preferably have a T2 relaxivity of at least 150 (mM·s)−1, even more preferably of at least approx. 160, 170, 180, 190, 200, 210, 220, 230, 240, 250, 260, 270, 280, 290, 300, 310, 320, 330, 340, 350, 400, 450, 500, 550, 600, 650, 700, 750, 800, 850, 900, 950 or 1000 (mM·s)−1. The T2 relaxivity of the particles is preferably determined at a magnetic field strength of 1 Tesla in an aqueous colloidal solution of the particles. Suitable measurement methods are known to the person skilled in the art and are also disclosed for example in the appended examples. In one preferred embodiment of the method according to the invention, the overall particle diameter varies within a range from approx. 30 nm to approx. 2 μm. However, even more preference is given to the use of particles having an overall diameter in a range from approx. 40 to approx. 500 nm, even more preferably in a range from approx. 45 nm to approx. 300 nm, yet more preferably in a range from approx. 50 nm to approx. 200 nm. As already mentioned, the overall diameter of the particles according to the invention can be determined by a number of direct and indirect methods known in the prior art, which include for example electron microscopy and dynamic light scattering. Preferred lower limits of the diameter of the iron oxide core contained in the particle are 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 85, 90, 95, 100 nm. Preferred upper limits of the diameter are 500, 490, 480, 470, 460, 450, 440, 430, 420, 410, 400, 390, 380, 370, 360, 350, 340, 330, 320, 310, 300, 290, 280, 270, 260, 250, 240, 230, 220, 210, 200, 190, 180, 170, 160, 150, 140, 130, 120, 110, 100, 95, 90, 85 and 80 nm. All combinations of the abovementioned upper and lower limits are possible in order to define the preferred range of the particle sizes that can be produced by the method according to the invention, provided that the value of the upper limit is greater than the value of the lower limit, for example 40 to 400 nm, 50 to 200 nm, etc. Preferred particles produced according to the invention have an iron oxide core diameter in a range from approx. 25 nm to approx. 500 nm. Even more preferably, the diameter of the iron oxide core lies in a range from approx. 30 nm to approx. 200 nm, yet more preferably from approx. 35 nm to approx. 80 nm.

Another subject matter of the present invention comprises particles which can be produced by a method according to the invention.

Another subject matter of the present invention comprises a composition, wherein at least 2% of the particles contained in the composition, which particles contain a magnetic iron oxide core in a pharmaceutically acceptable shell, are particles according to the invention or particles produced by the method according to the invention. In preferred embodiments, the proportion of particles according to the invention or of particles produced according to the invention is higher and is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% or 100%. The suitability of the compositions according to the invention for MPI methods increases as the proportion of particles according to the invention or of particles produced according to the invention increases.

Another subject matter of the present invention is a composition, wherein the particle diameter of the particles according to the invention or of the particles produced by the method according to the invention lies within a range of 10% around the averaged particle diameter in respect of at least 50% of the particles. An increase in the homogeneity of the size distribution and thus of the signal generated by the respective particle is preferred. In this connection, it is particularly preferred if at least 55%, at least 60%, 65%, 70%, 75%, 80%, 90%, 95% of the particles according to the invention or of the particles produced by the method according to the invention have a particle diameter which lies in a range of 10% around the averaged particle diameter. In this connection, the averaged particle diameter has the meaning already explained above, that is to say the particle diameter is the averaged diameter of all the particles according to the invention or particles produced according to the invention that are contained in the composition.

Another subject matter of the present invention is a fluid which contains a composition according to the invention. Suitable fluids are aqueous solutions which are preferably buffered to a physiological pH and which possibly contain salts, sugar, etc., particularly when they are intended for parenteral application. The fluids possibly contain additives such as preservatives, stabilizers, detergents, flavorings, excipients, etc. A plurality of substances which can be added to diagnostic solutions depending on the route of application are known to the person skilled in the art. These additives may be added to the fluids according to the invention without any exception apart from incompatibilities with the particles according to the invention. It is preferred if the fluid is in the form of a stabilized colloidal solution.

Another subject matter of the present invention is the use of a composition according to the invention or of a fluid according to the invention in order to produce a diagnostic means for use in magnetic particle imaging (MPI) to diagnose diseases. It is preferred here if the diseases are selected from the group consisting of proliferative diseases, in particular tumors and metastases, inflammatory diseases, autoimmune diseases, diseases of the digestive tract, arteriosclerosis, apoplexy, infarction, pathological changes in the vascular system, the lymphatic system, the pancreas, the liver, the kidneys, the brain and the bladder, and also diseases affecting electrical stimulus transmission and neurodegenerative diseases. The particles used here are preferably the particles disclosed above as being preferred or particularly preferred.

Another subject matter of the present invention is the use of a composition, wherein at least 2% of the particles contained in the composition comprise, in a pharmaceutically acceptable shell, a magnetic iron oxide core having a diameter of 20 nm to 1 μm and with an overall particle diameter/core diameter ratio of less than 6, in order to produce a diagnostic means for use in magnetic particle imaging (MPI) to diagnose diseases. It is preferred here if the diseases are selected from the group consisting of proliferative diseases, in particular tumors and metastases, inflammatory diseases, autoimmune diseases, diseases of the digestive tract, arteriosclerosis, apoplexy, infarction, pathological changes in the vascular system, the lymphatic system, the pancreas, the liver, the kidneys, the brain and the bladder, and also diseases affecting electrical stimulus transmission and neurodegenerative diseases. Preferred iron oxide core diameters of these particles are the diameters mentioned above, but also include iron oxide cores having a diameter of at least 5 nm, at least 6, 7, 8, 9, 10, 11, 12, 13, 14, 15, 16, 17, 18, 19, 20, 21, 22, 23, 24, 25, 26, 27, 28, 29 and 30 nm. The particles which can be used in this use according to the invention can be produced by the method described above.

It is particularly preferred if the particles are detected by a method comprising the following steps:
(i) generating a magnetic field with a spatial course of the magnetic field strength which is such that a first partial region with a low magnetic field strength and a second partial region with a higher magnetic field strength are obtained in the examination area,
(ii) changing the spatial position of the two partial regions in the examination area so that the magnetization of the particles changes locally,
(iii) recording signals which are dependent on the magnetization in the examination area that has been affected by this change,
(iv) evaluating the signals so as to obtain information about the spatial distribution of the magnetic particles in the examination area.

In this method and in this arrangement, a spatially inhomogeneous magnetic field is generated in the examination area. In the first partial region, the magnetic field is so weak that the magnetization of the particles differs to a greater or lesser extent from the external magnetic field, that is to say is not saturated. In the second partial region (that is to say in the rest of the examination area outside the first part), the magnetic field is strong enough to keep the particles in a state of saturation. The magnetization is saturated when the magnetization of almost all the particles is oriented in approximately the direction of the external magnetic field, so that with any further increase in the magnetic field the magnetization increases to a much lesser extent there than in the first partial region given a corresponding increase in the magnetic field.

The first partial region is preferably a spatially coherent region; it may be a point-shaped region but may also be a line or a surface area. Depending on the configuration, the first partial region is spatially surrounded by the second partial region.

By changing the position of the two partial regions within the examination area, the (overall) magnetization in the examination area changes. The change in the spatial position of the partial regions may for example be effected by means of a temporally changing magnetic field. If the magnetization in the examination area or physical parameters influenced thereby are measured, information can be derived therefrom about the spatial distribution of the magnetic particles in the examination area.

To this end, for example, the signals induced in at least one coil due to the temporal change in the magnetization in the examination area are received and evaluated. If a temporally changing magnetic field acts on the examination area and on the particles in a first frequency band, then, of those signals received by the coil, only those signals which contain one or more higher frequency components than those of the first frequency band are evaluated. These measured signals are generated since the magnetization characteristic of the particles usually does not run in a linear manner.

For further explanations concerning the method and the arrangement, reference is made to DE 101 51 778. Since said document describes the method and the arrangement in detail, the content of DE 101 51 778 is hereby fully incorporated by way of reference at this point.

In one preferred embodiment, the invention relates to the use of a composition or fluid according to the invention, wherein the arrangement for carrying out the detection comprises the following means:
a) means for generating a magnetic field with a spatial course of the magnetic field strength which is such that a first partial region with a low magnetic field strength and a second partial region with a higher magnetic field strength are obtained in the examination area,
b) means for changing the spatial position of the two partial regions in the examination area so that the magnetization of the particles changes locally,
c) means for recording signals which are dependent on the magnetization in the examination area that has been affected by this change in spatial position,
d) means for evaluating the signals so as to obtain information about the spatial distribution of the magnetic particles in the examination area.

Since the particles which can be used according to the invention permit a particularly high spatial resolution, they can be used in the diagnosis of proliferative diseases, and in particular early phases of such diseases. The proliferative disease is preferably selected from the group consisting of a tumor, a precancerous condition, a dysplasia, an endometriosis and a metaplasia.

Further preferred diseases which can be diagnosed using particles which can be used according to the invention include autoimmune diseases which are selected from the group consisting of rheumatoid arthritis, inflammatory bowel disease, osteoarthritis, neuropathic pain, alopecia greata, psoriasis, psoriatic arthritis, acute pancreatitis, allograft rejection, allergies, allergic inflammation in the lungs, multiple sclerosis, Alzheimer's disease, Crohn's disease, and systemic lupus erythematosus.

In another embodiment of the present invention, the particles according to the invention and particles produced according to the invention can be used in a method for local heating using magnetic particles, particularly preferably to generate hyperthermia. For further explanations concerning the method and arrangement, reference is made to WO2004/018039. Since said document describes the method and the arrangement in detail, the content of WO2004/018039 is hereby fully incorporated by way of reference at this point.

In another embodiment, the novel particles according to the invention and particles produced according to the invention can be used as T2-increasing contrast agents in Magnetic Resonance Imaging (MRI). The particles can be used as MRI contrast agents to diagnose diseases and pathological changes selected from the group consisting of proliferative diseases, inflammatory diseases, autoimmune diseases, diseases of the digestive tract, arteriosclerosis, apoplexy, infarction, pathological changes in the vascular system and the heart, the lymphatic system, the pancreas, the liver, the kidneys, the brain and the bladder, and also diseases affecting electrical stimulus transmission and neurodegenerative diseases. Particular preference is given to the use of the particles as MRI contrast agents for imaging the liver or spleen and to diagnose diseases of these organs. The particles can also be used as MRI contrast agents for angiography.

The following examples explain the invention without limiting it to the specific embodiments. The person skilled in the art is capable of finding a plurality of variations to the quantities and temperatures specified in the experiments, all these variations lying within the scope of the invention as defined by the appended claims.

### DESCRIPTION OF THE FIGURES AND IMAGES

FIG. 1 Shows the frequency distribution of the size of the cores of the particles produced, wherein the frequency varies between 0 and 1. (corresponds to 0% to 100%).

FIG. 2 The image marked A shows a TEM image of a composition according to Example 1c) (residue 1). A few non-spherical cores with a mean diameter of approximately 35 nm can be seen. The image marked B shows a high-resolution TEM of a large core of a preparation according to 1c) (residue 1). An accumulation of small monocrystals can be seen, which have aggregated to form a larger polycrystal.

FIG. 3 Results of an MPI examination using three different commercially available Resovist® batches. The Fourier amplitude of the signal obtained (y axis) at the specified multiples of the drive field frequency (x axis) is shown as the result.

FIG. 4 The image marked A shows a phantom for generating images using an arrangement and a method according to DE 101 51 778. This phantom contains a plurality of cavities (shown in the image as dark spots), which are filled with Resovist®. Image B shows an image of this phantom, wherein the cavities filled with Resovist® appear as light areas.

FIG. 5 The result of an MPI measurement using the preparation according to Examples 1b) and 1c) (two different averaged overall diameters) is shown in comparison to commercially available Resovist®. The Fourier amplitude of the signal obtained (y axis) at the specified multiples of the drive field frequency (x axis) is shown as the result.

### EXAMPLES

### Example 1

### Production of the Magnetic Iron Oxide Particles

### Example 1a

### Production of the Starting Particle Dispersion

105 g of carboxydextran (CDX) having an intrinsic viscosity of 0.050 dl/g are dissolved in 350 ml of water. Under a feed of nitrogen, an aqueous solution of 13.6 g iron(II) chloride tetrahydrate and 140 ml 1 M iron(III) chloride solution (corresponding to 37.8 g iron(III) chloride hexahydrate) is added thereto. Then, 242 ml of 3 N NaOH solution are added thereto, with stirring, over 15 min, and the mixture is heated to 80° C. The pH is adjusted to 7.0 by adding 6 N HCl. The mixture is further stirred at reflux for 1 h.

After cooling, the sample is centrifuged (2100 g/30 min). Ethanol is added in a proportion corresponding to 78% of the volume of the supernatant of the precipitate. The sample is again centrifuged (2100 g/10 min). The precipitate is dissolved in water and dialyzed against water (16 h). The dialysate is adjusted to pH 7.2 by means of NaOH and is concentrated under reduced pressure. The concentrate is filtered through a membrane filter (pore size: 0.2 μm) in order to obtain 186 ml of a suspension of carboxydextran-stabilized iron oxide particles. The production method corresponds to the production method for particles which are contained in Resovist®.

Iron concentration: 52 mg/ml (iron content: 91%), particle diameter of the magnetic iron oxides: 9 nm, overall particle diameter: 61 nm, water-soluble carboxypolysaccharide/iron weight ratio 1.15, magnetization at 1 Tesla: 98 emu/g of iron, T2 relaxivity: 240 (mM sec)−1

### Example 1b

### Production of Small Particles

A magnetic filter is composed of an annular magnet (NE 1556, IBS Magnet Berlin, external diameter 15 mm, internal diameter 5 mm, height 6 mm) and a separation chamber arranged in the interior volume of the annular magnet. The separation chamber consists of a wall made of plastic, and is filled with iron shot (diameter approx. 0.3 mm). 0.8 ml of a dispersion of iron oxide particles (according to Example 1a) having an iron content of 500 mmol/l and a T2 relaxivity of approximately 240 (mM·sec)−1 is filtered through the magnetic filter by means of hydrostatic pressure. The T2 relaxivity of the filtrate thus obtained is 41 (mM·sec)−1.

### Example 1c

### Production of the Novel Magnetic Iron Oxide Particles

Following the recovery of the iron oxide particles filtered according to Example 1b), the residue in the magnetic filter is obtained by rinsing once the magnet has been switched off. The resulting particle suspension (residue 1) has a T2 relaxivity of 293 (mM·sec)−1 and is characterized according to Examples 2, 3, 4 and 5.

Upon carrying out the filtration according to Example 1b) a second time, but this time with a weaker magnetic field, the residue (residue 2) obtained after switching off the magnet has a T2 relaxivity of 388 (mM·sec)−1.

### Example 2

### Determination of the Overall Particle Size by Means of Dynamic Light Scattering

Using a particle sizer from the company Malvern (ZetaSizer 3000 Hs a), the mean diffusion coefficients (intensity-weighted) of the particles are measured and the mean hydrodynamic diameter is calculated therefrom. This method represents one possibility for determining an averaged overall particle diameter. However, in this case, the diameter of the iron oxide core plus the hydrated shell is determined.

The results are summarized in the following Table 1:

TABLE 1

| | Averaged overall particle |
|---|---|
| | diameter [nm] |
| Example 1a) starting | 61 |
| dispersion | |
| (particles for Resovist ®) | |
| Example 1b) | 18 |
| filtrate | |
| Example 1c) | |
| residue 1 | 65 |
| residue 2 | 71 |

### Example 3

### Determination of the Core Size by Means of Electron Microscopy

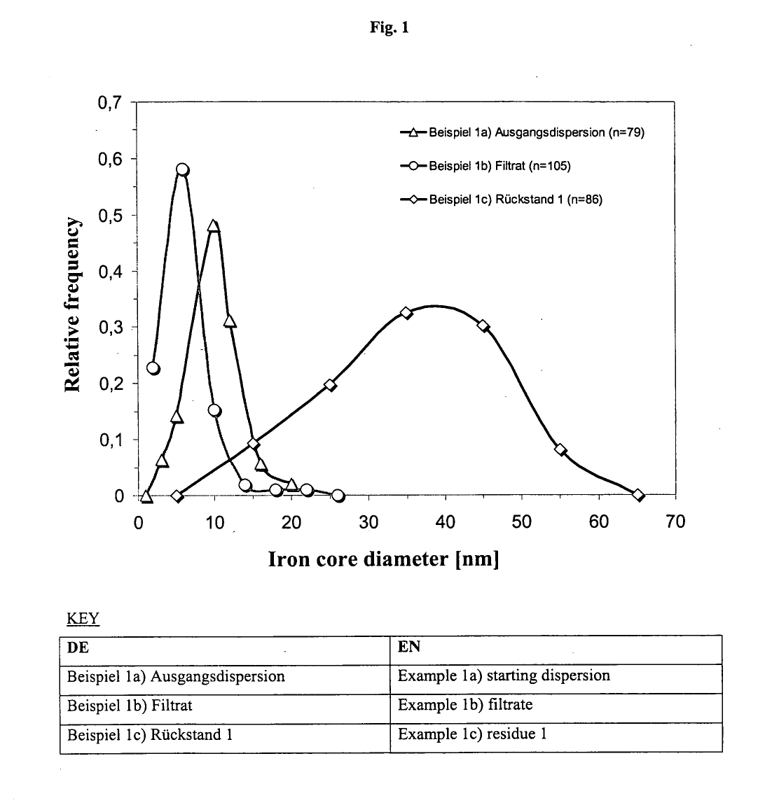

Using transmission electron microscopy (TEM), the contrast-rich iron oxide cores, since these have a higher electron density than the shell, are enlarged and photographed. The size of the particles imaged in this way is measured and the actual core size is calculated by way of the enlargement factor. 50 to 100 particles are counted and the results are plotted in a histogram. The averaged core diameter (number-weighted) is also calculated. The results are summarized as a histogram in FIG. 1 and in table formin Table 2.

TABLE 2

| | Averaged core diameter |
|---|---|
| | [nm] |
| Example 1a) starting | 9 |
| dispersion | |
| (particles for Resovist ®) | |
| Example 1b) | 6 |
| filtrate | |
| Example 1c) | |
| residue 1 | 35 |
| residue 2 | not determined |

### Example 4

### Determination of Overall Diameter/Core Ratio

The quotient is formed from the diameters according to Example 2 and the core sizes according to Example 3 and is summarized in the following Table 3.

TABLE 3

| | Averaged overall | Averaged core | Overall |
|---|---|---|---|
| | particle diameter | diameter | diameter/core |
| | [nm] | [nm] | ratio |
| Example 1a) | 61 | 9 | 6.78 |
| starting | | | |
| dispersion | | | |
| Example 1b) | 18 | 6 | 3.00 |
| filtrate | | | |
| Example 1c) | | | |
| residue 1 | 65 | 35 | 1.86 |
| residue 2 | 71 | not determined | — |

### Example 5

### Identification of the Polycrystallinity by Means of Electron Microscopy and High-Resolution Electron Microscopy

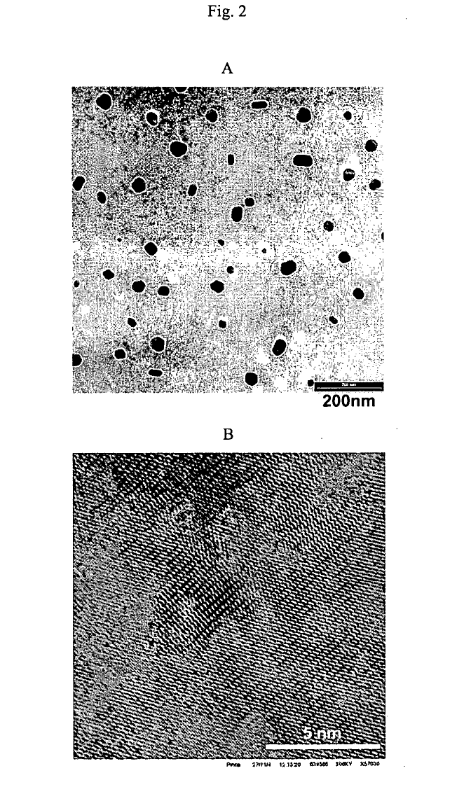

The polycrystallinity of the particles was determined by electron microscopy and high-resolution electron microscopy according to standard methods such as, for example, Transmission Electron Microscopy (TEM) on a CM2000 FEG (Philips) microscope at 200 kV (HRTEM). The samples were deposited on a perforated carbon film on a copper grid. The particles of residue 1 were examined in this way. The results are shown at different resolutions in FIGS. 2A and 2B.

### Example 6

### MPI Using Magnetic Iron Oxide Particles

### Example 6a

### Preparation of Samples for MPI Experiments

A hole having a diameter of 0.5 mm is drilled into a PVC plate (thickness 1 mm, lateral dimension approximately 2×2 mm²). One side is closed with sticky tape. Then, using a thin copper wire (0.2 mm coated), substance is placed dropwise into the hole until the latter is completely full. The substance is used in undiluted form. The open side of the hole is closed with sticky tape and the slide is glued onto a glass-fiber-reinforced dipstick. The sample is then sealed with acrylate adhesive on all sides.

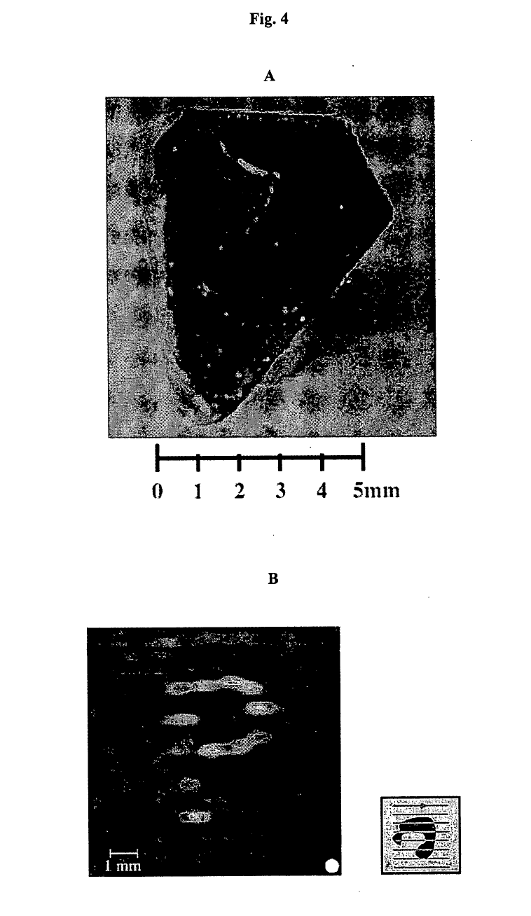

The sample is installed in an MPI scanner as described in DE 101 51 778 A1 and measured. Unlike the image generation as described in DE 101 51 788 A1, the sample is examined only with regard to the measured signal strength at different frequencies. To this end, a magnetic gradient field is used in the MPI scanner, as explained in FIG. 2 of DE 101 51 778 A1 and the associated description. The maximum gradient field strength is 3.4 T/m/μ0. An alternating magnetic field, as denoted H(t) in FIG. 4a of DE 101 51 778 A1, is superposed on this gradient field. The amplitude of the alternating field is 10 mT/μ0 in the direction of the maximum gradient of the gradient field, and the frequency is 25.25 kHz. The sample can be displaced mechanically in the MPI scanner, so that data can be acquired from different measuring points. At present, 52×52 measuring points are recorded, these being distributed over a surface area of 10×10 mm². The measurement time at each point is 0.4 s.

Of the signals recorded at the 52×52 measuring points, the signal value having the highest and the lowest signal strength is determined. These signal values are Fourier-transformed signals and are therefore complex values. Firstly, the difference between the real parts of the highest and lowest signal value and the difference between the imaginary parts of the highest and lowest signal value are formed. The square root of the sum of the squares of the two determined differences (y axis or vertical axis) at the specified multiples of the frequency of the alternating magnetic field (x axis or horizontal axis) is then plotted as the result.

### Example 6b

### MPI Using Resovist®

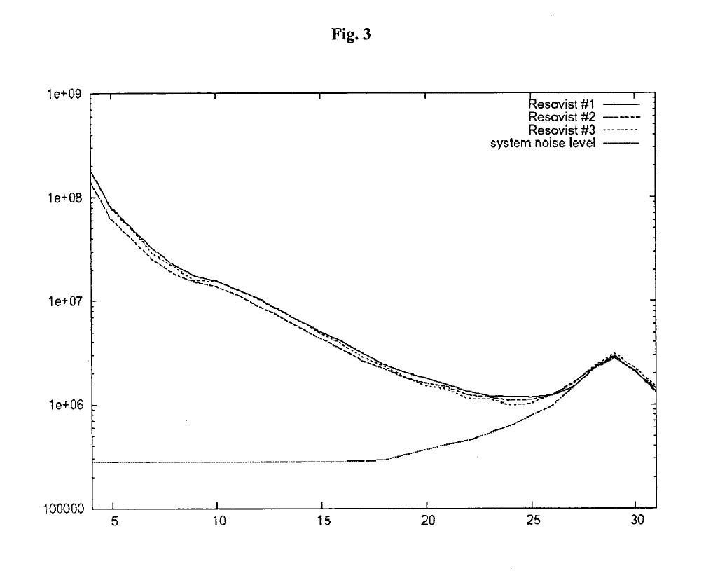

Resovist®, a commercially available product, is prepared and measured according to Example 6a). The iron concentration in Resovist® is 500 mmol Fe/l. The results are summarized in FIG. 3. A signal-to-noise ratio of up to approximately 25 times the drive field frequency was observed. Furthermore, reproducible results were obtained in independent experiments (3 different Resovist® batches). It was thus possible to demonstrate that Resovist is suitable for MPI.

### Example 6c

### MPI Using the Preparation According to Example 1b)

The iron concentration of a suspension according to Example 1b) is adjusted to 500 mmol Fe/l and then the preparation is prepared and measured according to Example 6a).

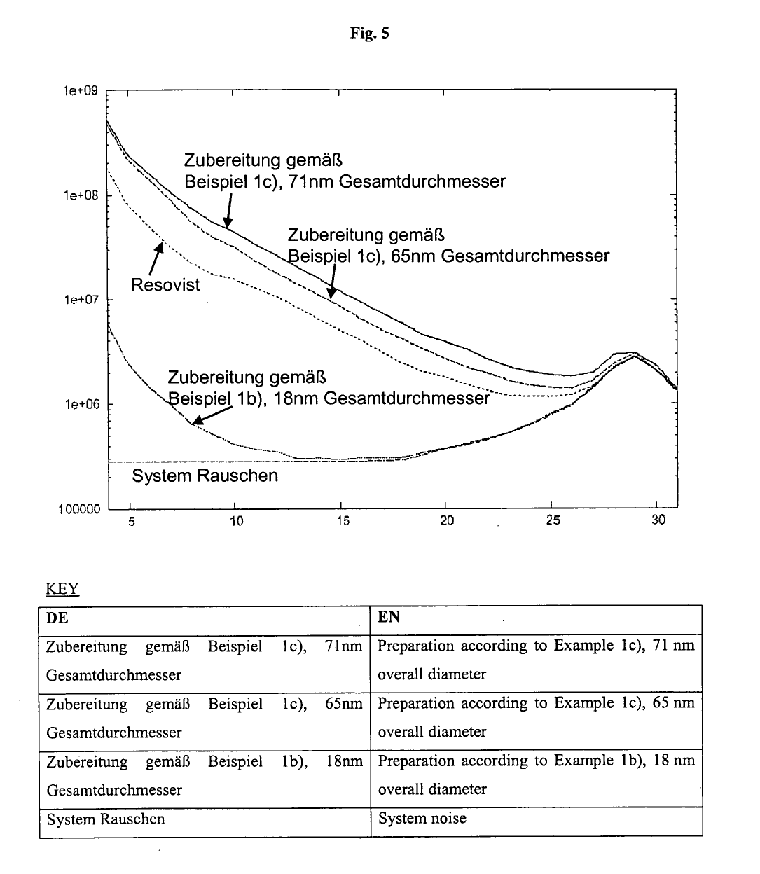

The results are shown in FIG. 5 together with the results of the comparison sample and the results of Example 6d).

It was found that the composition according to Example 1b) (filtrate) exhibits a considerably worse signal-to-noise ratio than the composition according to Example 1a) (starting dispersion) or Resovist®.

### Example 6d

### MPI Using Preparations According to Example 1c

The iron concentration of the suspensions according to Example 1c) (residue 1 and residue 2) is adjusted to 500 mmol Fe/l and then the preparations are prepared and measured according to Example 6a). The results are shown in FIG. 5 together with the results of the comparison sample and the results of Example 6c).

It was found that the compositions containing the novel particles according to the invention—the preparation according to Example 1c)—exhibit better signal-to-noise ratios than the starting dispersion according to Example 1a) (and the comparison sample of a commercially available MR contrast agent, Resovist®) and the filtrate according to Example 1b).

The novel particles according to the invention contained in the preparation according to Example 1c) are accordingly suitable for improved MPI.

### Example 6e

### MPI Imaging Using Resovist®

FIG. 4 shows in image A the image of a phantom for generating images using an arrangement and a method according to DE 101 51 778. This phantom contains a plurality of cavities (shown as dark spots in the image), which are filled with Resovist®. FIG. 4 shows in image B an image of this phantom, wherein the cavities filled with Resovist® appear as light areas.

### Example 7

### Synthesis of Chelator Iron Oxide Particles and Coupling of Multi-His-L-Selectin (MECA79) as In Vivo Contrast Agent

The text below describes the coupling of NTA (nitrilotriacetic acid derivative; α-N-[bis-carboxymethyl]lysine) to carboxydextran-stabilized magnetic iron oxide particles produced according to Example 1).

For this purpose, the carboxydextran-stabilized magnetic iron oxide particles are oxidized in aqueous solution with a 31-fold particle excess of sodium periodate (based on the carboxydextran) for 30 min with stirring in the dark at room temperature (RT). The sodium periodate is then separated quantitatively via gel filtration. The dextran magnetites are eluted in phosphate buffer (0.1 M phosphate buffer pH 7.0). NTA is then added to the oxidized dextran magnetites and the mixture is incubated in the dark for 2 h at RT with occasional shaking. In the process, NTA can be coupled in excess to the dextran magnetites. 1/10 volume of the reducing agent dimethylborane (150 mM in H₂O) are then added, and the mixture is incubated in the dark for a further 2 h at RT with occasional shaking. The last step is repeated, followed by an incubation at 4° C. overnight. The separation of the unbound NTA from the NTA that has bound to the surface of the particles is effected by way of gel filtration or ultrafiltration. The particles are eluted in PBS or in 0.1 M HEPES (in each case pH 7.0-7.4) and stabilized by adding 5 mg/ml of carboxydextran (final concentration). The particles are sterile-filtered, and sodium azide in a final concentration of 0.1% is added. The iron content of the suspension and the mean particle size are then determined.

In order to check the efficiency with which the NTA couples to the surface of the particles, the particles are firstly incubated with 10 mM EDTA in PBS or 0.1 M HEPES for 1 h at RT with occasional shaking. The EDTA is then removed via gel filtration or ultrafiltration, and the sample is incubated with Co2+, Ni2+ or comparable bivalent ions which are complexed by the chelator.

Excess ions are then separated from the particles via gel filtration or ultrafiltration. The number of NTA molecules that have bound to the particle surface can be determined by an ICP measurement of the bound ions, subtracting the ions which bind to unmodified dextran magnetites.

### Example 8

### Coupling of Multi-His-L-Selectin to NTA Dextran Magnetites

The NTA-carrying dextran magnetites are incubated firstly with Ni2+ ions (or similar ions) and then with multi-His-tagged selectin molecules in PBS or 0.1 M HEPES with 0.2% milk (in order to reduce non-specific binding) for 10 min at RT. Unbound selectin molecules are removed via suitable ultrafiltration units or via magnetic columns (Miltenyi Biotec) with an applied magnetic field. The resulting contrast agent constructs are checked in vitro for their binding capability, for example in the frozen section of peripheral mouse lymph nodes, and can then be used for in vivo experiments for imaging.

### Example 9

### Coupling of Streptavidin to Magnetic Iron Oxide Particles According to Example 1

The suspensions according to Example 1) are purified by means of at least 3-fold sedimentation in the ultracentrifuge and equal-volume take-up with 0.02% TritonX100 sodium acetate buffer solution (pH 4.5). 1 ml of the purified suspension is treated with 1 ml of a 2% streptavidin solution and stirred for 60 minutes at 4° C. 10 mg of EDC are then added. The pH value is monitored during this process. Should the pH deviate from 4.5+/−0.2, it must be readjusted using 0.01N HCl or 0.01N NaOH.

Incubation is continued for approx. 16 hours at 4° C. with stirring, and then is ended by a 15-minute incubation with 1 M ethanolamine. The magnetic iron oxide particles to which streptavidin has bound are separated from the unbound protein and from the byproducts by means of multiple centrifugation.

The success of the coupling operation is demonstrated by means of an aggregation assay by adding multiple biotin-modified BSA. Following the addition of biotin-BSA-aggregated streptavidin, functionalized iron oxide particles lead to visible aggregates whereas untreated iron oxide particles on the other hand do not exhibit any aggregation and thus remain stable in dispersion.

A quantitative conclusion concerning the coupling success can be obtained by means of Surface Plasmon Resonance (BioCore, BioCore2000) on immobilized biotin-BSA.

### Example 10

### Binding of MECA79 Antibody to Streptavidin-Functionalized Magnetic Iron Oxide Particles According to Example 9

The magnetic iron oxide particles to which streptavidin has bound according to Example 9) are purified by two-fold centrifugation against Hepes buffer/TritonX100 solution 0.01%, buffered and concentrated. The purified microcapsules, which now bind biotin, are incubated for 1 hour with 1 mg of biotinylated MECA79 antibody and then washed.

In the same way, control particles can be produced using a biotinylated isotype IgM antibody (e.g. clone R4-22).

50% of the antibody quantities used are bound to the microcapsules (BioCore measurement: saturation column with anti-IgM-FITC antibodies). The MECA79 antibody recognizes the “peripheral node adressin”, a ligand group which is found only on the high endothelial venules of the peripheral and mesenteric lymph nodes.

### Example 11

### Coupling of Anti-Mouse CD105 Antibodies to Magnetic Iron Oxide Particles

In a manner analogous to Example 10), biotinylated anti-mouse CD105 antibodies are bound to streptavidin-functionalized magnetic iron oxide particles according to Example 9). The CD105 antibody recognizes angiogenesis-specific receptors and can be used for the image-assisted diagnosis of tumors.

### Example 12

### Coupling of Anti-Mouse ICAM-1 Antibodies to Magnetic Iron Oxide Particles

In a manner analogous to Example 10), biotinylated anti-mouse ICAM-1 antibodies are bound to streptavidin-functionalized magnetic iron oxide particles according to Example 9). The ICAM-1 antibody recognizes centers of inflammation for example in the experimental autoimmune encephalomyelitis (EAE) model in mice. The EAE model is used as an in vivo disease model for multiple sclerosis.

Without further elaboration, it is believed that one skilled in the art can, using the preceding description, utilize the present invention to its fullest extent. The following preferred specific embodiments are, therefore, to be construed as merely illustrative, and not limitative of the remainder of the disclosure in any way whatsoever.

In the foregoing and in the following examples, all temperatures are set forth uncorrected in degrees Celsius and, all parts and percentages are by weight, unless otherwise indicated.

The entire disclosure of all applications, patents and publications, cited herein and of corresponding European application No. 05014058.1, filed Jun. 29, 2005 is incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. A particle comprising, in a pharmaceutically acceptable shell, a magnetic iron oxide core having a diameter of 20 nm to 1 μm, characterized in that the overall particle diameter/core diameter ratio is less than 6.

2. A particle according to claim 1, characterized in that the pharmaceutically acceptable shell stabilizes the colloidal solution.

3. A particle according to claim 1, characterized in that the pharmaceutically acceptable shell comprises a synthetic polymer or copolymer, a starch or a derivative thereof, a dextran or a derivative thereof, a cyclodextran or a derivative thereof, a fatty acid, a polysaccharide, a lecithin or a mono-, di- or triglyceride or a derivative thereof or mixtures thereof.

4. A particle according to claim 3, wherein
(i) the synthetic polymer or copolymer is selected from the group consisting of polyoxyethylene sorbitan esters, polyoxyethylene and derivatives thereof, polyoxypropylene and derivatives thereof, nonionic surfactants, polyoxyl stearates (35-80), polyvinyl alcohols, polymerized sucrose, polyhydroxyalkyl methacrylamides, lactic acid and glycolic acid copolymers, polyorthoesters, polyalkylcyanoacrylates, polyethylene glycol, polypropylene glycol, polyglycerols, polyhydroxylated polyvinyl matrices, polyhydroxyethyl aspartamides, polyamino acids, styrene and maleic acid copolymers, polycaprolactones, carboxypolysaccharide, and polyanhydrides;
(ii) the starch derivative is selected from the group consisting of starch 2-hydroxymethyl ether and hydroxyethyl starch;
(iii) the dextran or derivative thereof is selected from the group consisting of galactosylated dextran, lactosylated dextran, aminated dextran, dextran containing SH groups, dextran containing carboxyl groups, dextran containing aldehyde groups, biotinylated dextran;
(iv) the cyclodextrin is selected from the group consisting of beta-cyclodextrin and hydroxypropyl cyclodextrin;
(v) the fatty acid is selected from the group consisting of sodium lauryl sulfate, sodium stearate, stearic acid, sorbitan monolaurate, sorbitan monooleate, sorbitan monopalmitate and sorbitan monostearate.

5. A particle according to claim 1, characterized in that the magnetic iron oxide core comprises magnetite or maghemite or mixtures thereof.

6. A particle according to claim 1, characterized in that the iron oxide core contains at least five iron oxide monocrystals.

7. A particle according to claim 1, characterized in that the overall particle diameter is 30 nm to 2 μm.

8. A particle according to claim 1, characterized in that the diameter of the iron oxide core is 30 to 500 nm.

9. A particle according to claim 1, characterized in that the iron oxide core is polycrystalline.

10. A particle according to claim 1, characterized in that polyethylene glycol and/or polypropylene glycol residues are covalently or non-covalently bonded to the surface of the particle.

11. A method for producing particles which comprise, in a pharmaceutically acceptable shell, a magnetic iron oxide core having a diameter of 20 nm to 1 μm and with an overall particle diameter/core diameter ratio of less than 6, said method comprising the following steps:
(i) mixing an aqueous mixed iron salt solution, which contains an iron(II) salt and an iron(III) salt, with an aqueous alkali solution in the presence of a synthetic polymer or copolymer, a starch or a derivative thereof, a dextran or a derivative thereof, a cyclodextran or a derivative thereof, a fatty acid, a polysaccharide, a lecithin or a mono-, di- or triglyceride or a derivative thereof or mixtures thereof, until a colloidal solution of the particles is formed,
(ii) passing the particle-containing solution through a magnetic gradient field,
(iii) removing the magnetic gradient field,
(iv) and recovering the particles retained in the gradient field.

12. A method according to claim 11, characterized in that, in a further step, particles of a predetermined overall particle diameter are selected.

13. A particle which can be produced by a method according to claim 1.

14. A composition, characterized in that at least 2% of the particles contained in the composition, which particles contain a magnetic iron oxide core in a pharmaceutically acceptable shell, are particles according to claim 1.

15. A composition according to claim 14, characterized in that the particle diameter of the particles lies within a range of 10% around the averaged particle diameter in respect of at least 50% of the particles.

16. A fluid, characterized in that it contains a composition according to claim 14.

17. A fluid according to claim 16, characterized in that the fluid is in the form of a stabilized colloidal solution.

18. The use of a composition according to claim 14 in order to produce a diagnostic means for use in magnetic particle imaging (MPI) to diagnose a disease selected from the group consisting of proliferative diseases, inflammatory diseases, autoimmune diseases, diseases of the digestive tract, arteriosclerosis, apoplexy, infarction, pathological changes in the vascular system, the lymph system, the pancreas, the liver, the kidneys, the brain and the bladder, and also diseases affecting electrical stimulus transmission and neurodegenerative diseases.

19. The use of a composition, characterized in that at least 2% of the particles contained in the composition comprise, in a pharmaceutical acceptable shell, a magnetic iron oxide core having a diameter of 20 nm to 1 μm and with an overall particle diameter/core diameter ratio of less than 6, in order to produce a diagnostic means for use in magnetic particle imaging (MPI) to diagnose a disease selected from the group consisting of proliferative diseases, inflammatory diseases, autoimmune diseases, diseases of the digestive tract, arteriosclerosis, apoplexy, infarction, pathological changes in the vascular system, the lymph system, the pancreas, the liver, the kidneys, the brain and the bladder, and also diseases affecting electrical stimulus transmission and neurodegenerative diseases.

20. The use according to claim 18, wherein the particles are detected by a method comprising the following steps:
(i) generating a magnetic field with a spatial course of the magnetic field strength which is such that a first partial region with a low magnetic field strength and a second partial region with a higher magnetic field strength are obtained in the examination area,
(ii) changing the spatial position of the two partial regions in the examination area so that the magnetization of the particles changes locally,
(iii) recording signals which are dependent on the magnetization in the examination area that has been affected by this change,
(iv) evaluating the signals so as to obtain information about the spatial distribution of the magnetic particles in the examination area.

21. The use according to claim 20, characterized in that the arrangement for carrying out the detection comprises the following means:
a) means for generating a magnetic field with a spatial course of the magnetic field strength which is such that a first partial region with a low magnetic field strength and a second partial region with a higher magnetic field strength are obtained in the examination area,
b) means for changing the spatial position of the two partial regions in the examination area so that the magnetization of the particles changes locally,
c) means for recording signals which are dependent on the magnetization in the examination area that has been affected by this change in spatial position,
d) means for evaluating the signals so as to obtain information about the spatial distribution of the magnetic particles in the examination area.

22. The use according to claim 18, characterized in that the proliferative disease is selected from the group consisting of a tumor, a precancerous condition, a dysplasia, an endometriosis and a metaplasia.

23. The use according to claim 18, characterized in that the autoimmune disease is selected from the group consisting of rheumatoid arthritis, inflammatory bowel disease, osteoarthritis, neuropathic pain, alopecia areata, psoriasis, psoriatic arthritis, acute pancreatitis, allograft rejection, allergies, allergic inflammation in the lungs, multiple sclerosis, Alzheimer's disease, Crohn's disease, and systemic lupus erythematosus.
